(19) United States
(12) Patent Application Publication
(10) Pub. No.: US 2013/0184689 A1
(43) Pub. Date: Jul. 18, 2013

(54) Surgical Drain

(71) Applicants: Michael DeWolfe, Chicago, IL (US); Kelly Retzlaff, Chicago, IL (US)

(72) Inventors: Michael DeWolfe, Chicago, IL (US); Kelly Retzlaff, Chicago, IL (US)

(21) Appl. No.: 13/723,649

(22) Filed: Dec. 21, 2012

Related U.S. Application Data
(60) Provisional application No. 61/579,520, filed on Dec. 22, 2011.

(51) Int. Cl. A61M 27/00

(52) U.S. Cl. CPC A61M 27/00
USPC 604/541; 604/319

(57) ABSTRACT

A device is provided for removing fluid from a body cavity. The device is a catheter that can be securely anchored to a patient to improve comfort and prevent infections and contamination. To accomplish this, the catheter is provided with (1) an inflatable disk and (2) a securing ring. When the catheter reaches a proper location in the body cavity, the inflatable disk is inflated and comes into contact with an inner surface of the body cavity. To further secure the catheter into place, the securing ring is then moved into position against the epidermis. In this configuration, the tissue between the epidermis and the body cavity is engaged by both the inflatable disk and the securing ring. This ensures that the catheter remains in place and can continue to operate effectively while promoting patient comfort.

This application claims the benefit of U.S. Provisional Patent Application Ser. No. 61/579,520, filed Dec. 22, 2011.

### TECHNICAL FIELD

The technical field pertains to catheters that are used to remove fluids from a patient, and more particularly pertains to a catheter used to remove fluids from a patient while remaining stationary when in use to increase the comfort level of the patient and minimize the likelihood of contamination or infection.

### BACKGROUND

Any type of invasive medical procedure carries a variety of risks. When the skin is pierced by a needle or any other type of instrument to create an incision, great care must be taken to ensure that all areas near the location of the incision remain clean at all times. Accomplishing this in a medical setting can be quite complicated due to the direct exposure of the incision location to the surrounding environment, which can often be contaminated with various types of contaminants.

In many cases, a catheter must remain in place for an extended period of time. As a result, the chance of infection or contamination increases greatly due to the amount of time the incision remains exposed to its surrounding environment. Also, movement of a patient can further expose the incision to its surrounding environment and also cause the outer surface of the catheter, or other type of medical apparatus being used, to come into contact with the location of the incision. Anchoring mechanisms, such as tape, are commonly utilized in an attempt to secure a catheter in place. However, commonly used anchoring mechanisms often do not adequately secure the catheter in place when a patient moves, and must be replaced frequently. When the catheter is no longer secure, the chances of contamination or infection increase greatly. An inadequately secured catheter also must be replaced more often than a properly secured catheter because a catheter that is not secured will come into contact with more contaminants than one that is properly secured.

Current methods of securing the catheter also are often quite uncomfortable for a patient. When a catheter is in place for an extended period of time, the catheter, associated tubing, and/or the tape used to secure the catheter may cause discomfort for the patient. This may lead to the patient moving the equipment in an effort to increase his comfort level. When this occurs, the catheter may become dislodged from the skin or even become dislodged from the body cavity into which it has been inserted. All of these situations may cause serious problems and may greatly decrease the effectiveness of the catheter in draining fluids from a body cavity.

In light of the above, it is desirable to provide a catheter that minimizes the risk of infection and contamination of the entry site. It is also desirable to provide a catheter that remains securely in place for any period of time. Also, it is desirable to provide a catheter that is comfortable for a patient. Furthermore, it is desirable to provide an infusion system that is easy to use, is simple to manufacture and is comparatively cost effective.

### SUMMARY

In accordance with an exemplary embodiment, a device for removing fluid from a body cavity of a patient is provided. A tubular-shaped catheter is inserted into a particular body cavity of the patient. Once the distal end of the catheter reaches an appropriate location within the body cavity, tissue between the cavity wall and the epidermis is engaged between an inflatable disk on the catheter and a securing ring, also on the catheter, to secure, or anchor the device in place. The structural cooperation of the inflatable disk and the securing ring makes the process of removing fluid more comfortable for a patient and minimizes the likelihood of contamination or infection. This is accomplished because both the securing ring and the inflatable disk assist in stabilizing the catheter.

Structurally, an exemplary catheter is tubular-shaped and has a lumen, a proximal end, and a distal end configured for insertion into the body cavity of the patient. Also, the catheter has an inner surface and an outer surface. An exemplary embodiment includes an insert having a length “l” that is positioned inside the lumen of the catheter and is of the type often used in the pertinent art. This insert extends in a proximal direction from the distal end of the catheter, with the length “l” being determined based primarily on anatomical factors of the body cavity being drained. Furthermore, the insert may be constructed using either a fluted-round configuration, a flat-perforated configuration, or any other configuration that is appropriate for draining fluid from a body cavity. An additional component included in an exemplary embodiment is an inflatable disk having a diameter “D” and affixed to the catheter at a distance “d” from the distal end of the catheter. In an embodiment, the disk is affixed onto a particular location on the catheter just prior to insertion into a body cavity. In other embodiments, catheters may be produced with the disk affixed at a variety of standard lengths from the distal end of the catheter. For purposes of affixing the disk to the catheter, the disk is formed with a round hole in its center configured to allow the catheter to pass through the center of the disk. This disk is inflatable and may be selectively inflated to an inflated configuration and selectively deflated to a deflated configuration. Inflation (and deflation) is accomplished using an infusion of saline solution or any other appropriate fluid used in the pertinent art. To facilitate the inflation of the disk, an inflation line is provided that has a first end connected in fluid communication with the inflatable disk and a second end formed with an inflation port. As described herein, the inflation line extends between the inflatable disk and an inflation port inside the lumen of the catheter, with a portion of the inflation line extending outside of the catheter to allow for access to the inflation port. This accessibility allows for an inflation pump to be connected to the inflation port for inflating and deflating the inflatable disk as required.

In an exemplary embodiment, a securing ring is engaged with the catheter. This securing ring can be ring-shaped, but it also may be U-shaped, or have other shapes. The securing ring is formed with a plurality of securing taps that are used to establish contact with, or grip, the outer surface of the catheter. Both the securing ring and the securing tabs are envisioned as being constructed with antimicrobial material, such as antimicrobial plastic. The contact established with the catheter is tight enough to engage the ring with the catheter, but loose enough to allow the securing ring to be slidably moveable along the outer surface of the catheter. Various shapes can be used for the securing tabs, with triangular-shaped tabs being one exemplary embodiment. Furthermore, the securing ring may also be constructed with at least two needle insertion holes to further secure the ring to a patient using sutures if desired or necessary. These needles holes will also be constructed with an antimicrobial material. In addition to these components, the catheter is also constructed with an indicator notch that is either an indentation or a colored stripe that extends around the circumference of the outer surface of the catheter at a particular location. This particular location is chosen to ensure that the distal end of the catheter is situated in a desired location within the body cavity after insertion. In other words, the indicator notch verifies a proper insertion of the distal end of the catheter into the body cavity chosen for drainage. In an embodiment, the indicator notch is aligned with the epidermis of the patient when the catheter is situated properly in the body cavity selected for drainage. Because the notch is indented into the catheter, the notch can catch, or receive, the securing ring when the ring is moved towards the epidermis and reaches the position on the catheter where the securing ring will remain during drainage of the body cavity.

In order to ensure proper draining of the body cavity, the device described herein also may include a fluid collection chamber that is connected in fluid communication via a drainage line with the proximal end of the catheter. In addition, a suctioning device may be connected to the fluid collection chamber to assist in draining the body cavity.

In an operation of the device, a body cavity is selected for drainage. Depending on the characteristics of the body cavity (for instance, the depth beneath the epidermis or the location on the body), a catheter is chosen with an appropriate indicator notch. This means that the indicator notch will indicate that the distal end of the catheter has reached a proper position, or depth, within the body cavity to drain the body cavity appropriately. After the catheter with the appropriate indicator notch is selected, the distal end of the catheter is inserted into the body cavity through an incision to the appropriate position, as indicated by the indicator notch. Typically the indicator notch is aligned with the epidermis of the patient when the catheter is situated properly. After insertion to the appropriate position, the inflation pump is connected to the inflation port of the inflation line. Then, the inflatable disk is inflated from its first, or deflated, configuration, to its second, or inflated, configuration. After it is in the second configuration, the catheter is withdrawn slightly until the inflatable disk makes contact with the inner surface of the body cavity. This action ensures that the catheter cannot be removed, whether advertently or inadvertently. In order to further secure the catheter in place, the securing ring is moved down the outer surface of the catheter and into contact with the epidermis of the patient to create a tight seal. The securing ring can be taped down or sutured using the needle insertion holes provided on the securing ring. However, neither tape nor suturing may be required. With the combination of the inflatable disk in contact with the wall of the body cavity and the securing ring outside the body cavity against the epidermis, the catheter is effectively secured in place. This securing of the catheter in place will allow fluid to drain more effectively and provide minimal discomfort to the patent. As secured, the catheter can remain in this position for both short-term and long-term periods of time.

To begin drainage of the body cavity, the suctioning device is engaged. Once this occurs, fluid will flow out of the body cavity and into the catheter. For purposes of collecting the fluid, a fluid collection chamber may be connected by a drainage tube to the catheter. This fluid collection chamber remains in place until its capacity is reached. When the capacity is reached, the fluid collection chamber can be disconnected from the drainage tube and replaced with an empty fluid collection chamber.

Once the body cavity has been appropriately drained or has become ineffective in some way, the catheter can be removed from the body cavity. To begin removal, the suctioning device is shut off, and the securing ring is moved from its position in contact with the epidermis by sliding it along the outer surface of the catheter towards the proximal end of the catheter. Then, the inflatable disk is deflated using the inflation pump. When the disk is deflated and the securing ring is no longer in contact with the epidermis of the patient, the distal end of the catheter can be withdrawn from the body cavity. In most cases, the fluid collection chamber remains connected to the drainage tube throughout the process of removing the catheter from the body cavity.

### BRIEF DESCRIPTION OF THE DRAWINGS

FIG. 1 is a schematic drawing of a surgical drain in accordance with an embodiment;

FIG. 2 is a view of a surgical drain in an operational environment; and

FIG. 3 is a view of the securing ring.

### DETAILED DESCRIPTION

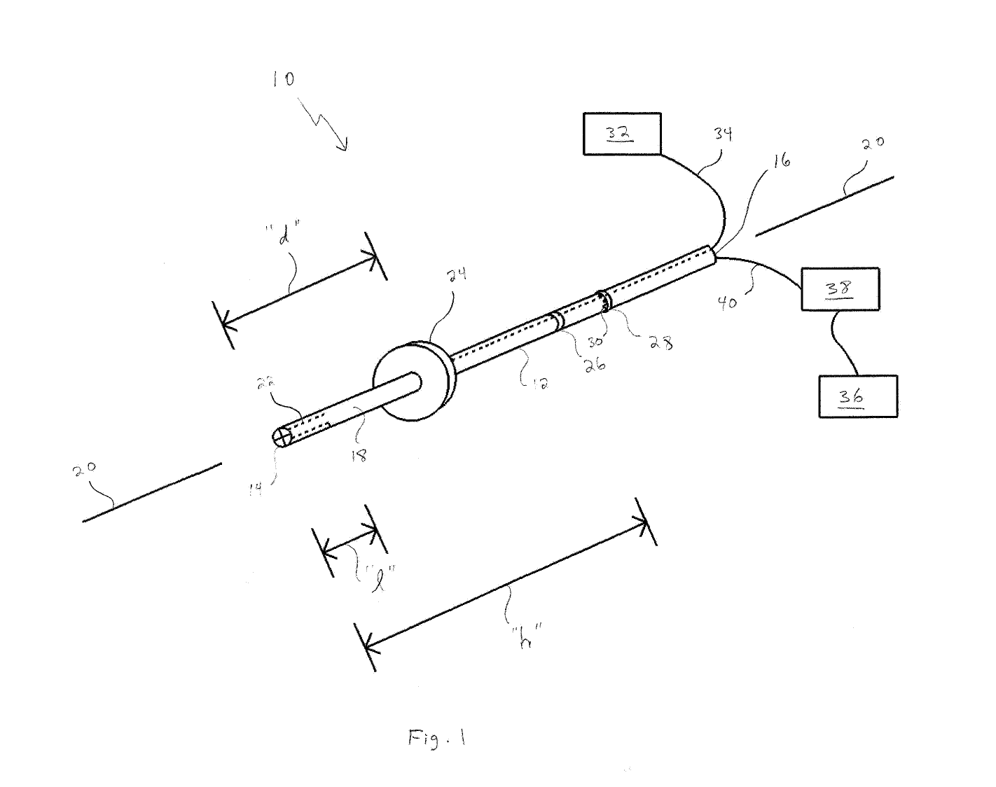

Referring initially to FIG. 1, a schematic drawing of a surgical drain in accordance with an exemplary embodiment herein is shown and generally designated 10. The surgical drain 10 is most often constructed of rubber or some other type of flexible material. It can be seen that the surgical drain 10 is comprised of a variety of structural components. The first structural component is a catheter 12 that can be made of either a flexible or a rigid material. The catheter 12 is of the type that is generally used in the pertinent art. In further detail, the catheter 12 is an elongated, hollow tube having a distal end 14, a proximal end 16, and a lumen 18 that extends from the distal end 14 to the proximal end 16 in the direction of an axis 20. It can be made of any type of plastic or other material commonly used to construct a catheter for use as a medical device intended for insertion into the human body. Another component of the surgical drain 10 is an insert 22 that extends in a proximal direction from the distal end 14 of the catheter 12 for a length “l.” The insert can be formed with a fluted-round configuration, a flat-perforated configuration, or any other configuration that is appropriate for promoting the drainage of fluid from a body cavity.

In FIG. 1, an inflatable disk 24 is affixed to the outer surface of the catheter 12 at a distance “d” from the distal end 14 of the catheter 12. The inflatable disk 24 can be made of silicone. It can be seen that the inflatable disk 24 is constructed with a hole at its center to allow the catheter 12 to pass through the inflatable disk 24. In FIG. 1, the inflatable disk 24 is in its inflated configuration. As shown, an indicator notch 26 is provided on the outer surface of the catheter 12 and is located at a distance “h” from the distal end 14 of the catheter 12. This indicator notch 26 can be an indentation in the outer surface of the catheter 12, a non-indented colored stripe that is a different color than the rest of the catheter 12, or an indented and colored stripe. For use with the indicator notch 26, a securing ring 28 is provided and is made of silicone or any other appropriate material. This securing ring 28 can move freely along the outer surface of the catheter 12 and can be seated into or onto the indicator notch 26 when the surgical drain 10 is inserted into a body cavity. The purpose of the securing ring 28 is to lock the catheter 12 in place against the epidermis when draining a body cavity. To promote contact between the securing ring 28 and the outer surface of the catheter 12, a plurality of securing tabs 30 are formed onto the securing ring for the purpose of gripping the catheter 12. These tabs, of which tab 30 is exemplary, are triangular in shape in an exemplary embodiment, but any shape may be used. In some cases, the securing ring 28 may not be formed with securing tabs 30. Tabs 30 are shown in more detail in FIG. 3.

Surgical drain 10 includes several additional components in FIG. 1. As shown, an external inflation pump 32 and an associated inflation line 34 are provided to inflate and deflate the inflatable disk 24. As shown, the inflation line 34 extends from the inflation pump 32 through the lumen 18 of the catheter 12 to the inflatable disk 24. The combination of the inflation pump 32 and the inflation line 34 are the illustrated means for inflating the inflatable disk 24 with saline solution or any other appropriate liquid. In FIG. 1, other components are connected to the surgical drain 10 to remove fluid from the body cavity. Specifically, a suctioning device 36, a fluid collection chamber 38, and a drainage line 40 are provided. The drainage line 40 extends from the fluid collection chamber 38 through the lumen 18 of the catheter 12 towards the insert 22.

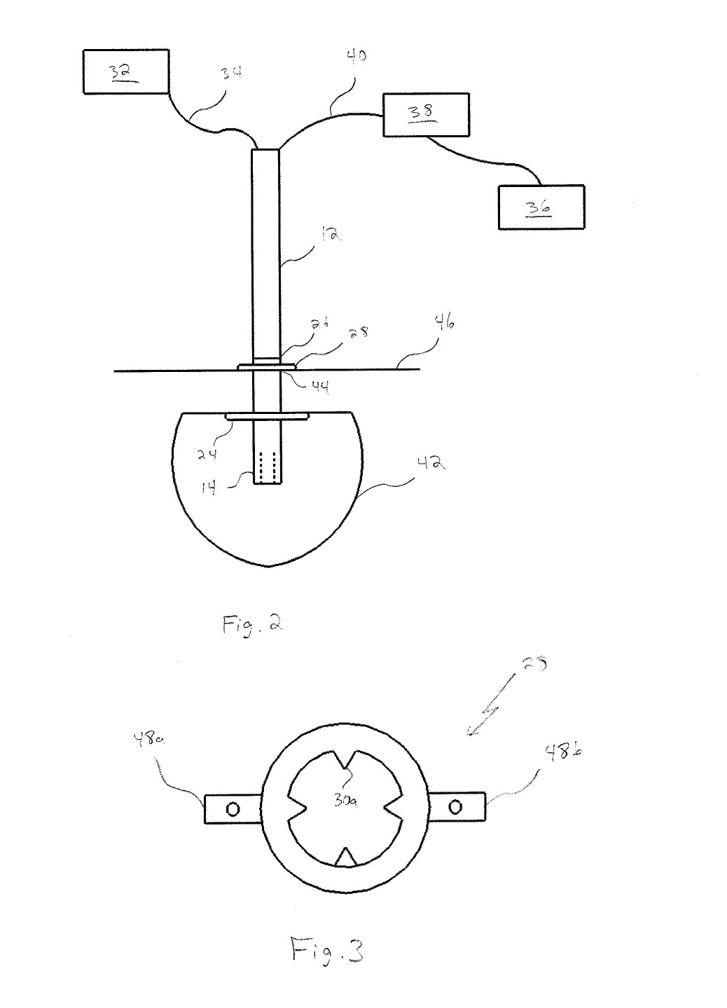

Referring now to FIG. 2, a view of the surgical drain 10 is shown in an operational environment and an operation of the surgical drain 10 can be described. As shown in FIG. 2, the surgical drain 10 is inserted into a body cavity 42. Specifically, the distal end of the catheter 12 has been inserted into an incision 44 on the epidermis 46 of a patient (not shown). During insertion of the catheter 12, the inflatable disk 24 is in a deflated position and is wrapped tightly against the outer surface of the catheter 12. This configuration allows the catheter 12 to reach an appropriate depth required for proper drainage of the body cavity 42. As the distal end 14 of the catheter 12 is inserted into the incision 44 on the epidermis 46, the securing ring 28 is able to freely slide along the outer surface of the catheter 12.

When the indicator notch 26 is flush with the epidermis 46, the appropriate depth has been reached by the distal end 14 of the catheter 12. At this point, the securing ring 28 is moved towards the distal end 14 of the catheter 12 until the securing ring 28 has reached the indicator notch 26, as shown. After the securing ring 28 has reached the indicator notch 26, the surgical drain 10 is anchored securely against the epidermis 46 and a tight seal has been formed between the epidermis 46 and the securing ring 26. Simultaneous with the engagement of the securing ring 28 with the epidermis 46, the inflation pump 32 is activated to move the inflatable disk 24 from the deflated position to the inflated position as shown in FIG. 2. Once the inflatable disk 24 is completely inflated, the inflatable disk 24 is secured tightly against the inner surface of the body cavity 42. A slight retraction of the catheter 12 will ensure that the inflatable disk 24 comes into contact with the inner surface of the body cavity 42.

With the inflatable disk 24 inflated and the securing ring 28 engaged with the indicator notch 26, the surgical drain 10 is securely anchored in place. At this point, suctioning device 36 can be activated to begin the drainage of the body cavity 42 into the fluid collection chamber 38.

After the body cavity 42 has been drained appropriately, to remove the surgical drain 10, the procedure is followed in reverse. This means the inflatable disk 24 is deflated, and the securing ring 28 is disengaged with the epidermis 46 and the indicator notch 26. The surgical drain 10 can now be removed from the body cavity 42.

Referring to FIG. 3, a more detailed view of an exemplary embodiment of the securing ring 28 is shown. The purpose of FIG. 3 is to more clearly illustrate the securing tabs, of which 30a is an example, and the needle insertion tabs 48a-b. The needle insertion tabs 48a-b can included as part of the securing ring 28 to further secure the securing ring 28 to the epidermis 46 (shown in FIG. 2) through the use of sutures or any other fastening means. This arrangement is exemplary and any number of securing tabs 30 and suturing tabs 48 can be implemented for use with the surgical drain 10.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the claimed subject matter in any way. Various changes may be made in the function and arrangement of elements described in an exemplary embodiment without departing from the scope set forth in the appended claims.

What is claimed is:

1. A device for draining fluid from a body cavity which comprises:
a tubular-shaped catheter formed with a lumen and having a proximal end and a distal end, wherein the catheter has an inner surface and an outer surface;
an insert having a length “l” positioned in the lumen of the catheter to extend in a proximal direction from the distal end of the catheter;
an inflatable disk having a diameter “D,” wherein the disk is attached to the catheter at a distance “d” from the distal end of the catheter;
an inflation line positioned inside the lumen of the catheter, wherein the inflation line has a first end connected to the inflatable disk and a second end formed with an inflation port;
an inflation pump for inflating the inflatable disk between a deflated configuration and an inflated configuration;
a securing ring engaged with the catheter and formed with a plurality of securing tabs, wherein the securing tabs establish contact between the securing ring and the outer surface of the catheter, and wherein the securing ring is slidably moveable on the outer surface of the catheter; and
an indicator notch formed on the catheter at a distance “h” from the distal end of the catheter, wherein the indicator notch indicates a proper insertion of the distal end of the catheter into the body cavity, for inflation of the disk into the inflated configuration and draining of fluid from the body cavity.

2. A device as recited in claim 1 wherein the insert is formed with fluted-round tubing to promote drainage of fluid from the body cavity.

3. A device as recited in claim 1 wherein the insert is formed with flat-perforated tubing to promote drainage of fluid from the body cavity.

4. A device as recited in claim 1 wherein the inflatable disk and the securing ring are formed with silicone.

5. A device as recited in claim 1 wherein the securing tabs are triangular in shape.

6. A device as recited in claim 1 wherein the catheter is formed with a flexible material.

7. A device as recited in claim 1 wherein the securing tab further comprises as least two needle insertion holes for suturing the securing tab to a surface.

8. A device as recited in claim 1 wherein the surgical drain is made of rubber.

9. A device as recited in claim 1 wherein the securing ring is positioned against the epidermis of a patient when the disk is in the inflated configuration.

10. A device as recited in claim 1 wherein the indicator notch is aligned with the epidermis of a patient when the disk is in the inflated configuration.

11. A device as recited in claim 1 wherein the securing ring and the plurality of securing tabs are made with an antimicrobial material.

12. A device as recited in claim 1 further comprising:
a fluid collection chamber, wherein the fluid collection chamber is connected in fluid communication by a drainage line with the proximal end of the catheter; and
a suctioning device, wherein the suctioning device is in fluid communication with the proximal end of the catheter and the fluid collection chamber to assist in draining fluid from the body cavity.

13. A surgical drain for draining fluid from a body cavity comprising:
a tubular-shaped catheter formed with a lumen and having a proximal end and a distal end, wherein the catheter has an inner surface and an outer surface;
an inflatable disk having a diameter “D,” wherein the disk is attached to the catheter at a distance “d” from the distal end of the catheter;
a securing ring engaged with the catheter, wherein the securing ring is slidably moveable on the outer surface of the catheter; and
an indicator notch formed on the catheter at a distance “h” from the distal end of the catheter, wherein the indicator notch indicates a proper insertion of the distal end of the catheter into the body cavity.

14. A surgical drain as recited in claim 13 further comprising an insert having a length “l” positioned in the lumen of the catheter to extend in a proximal direction from the distal end of the catheter.

15. A surgical drain as recited in claim 14 wherein the securing ring is formed with a plurality of securing tabs to establish contact between the outer surface of the catheter and the securing ring.

16. A surgical drain as recited in claim 13 wherein the indicator notch is indented into the outer surface of the catheter to receive the securing ring when the proper insertion of the distal end of the catheter into the body cavity has been accomplished.

17. A method for draining fluid from a body cavity using a tubular-shaped catheter formed with a lumen, wherein the catheter has an inner surface, an outer surface, a distal end, and a proximal end, and wherein the method comprises the steps of:
affixing an inflatable disk having a diameter “D” around the outer surface of the catheter, wherein the inflatable disk is deflated in a first configuration and inflated in a second configuration;
attaching a securing ring onto the outer surface of the catheter, wherein the securing ring is slidably moveable on the outer surface of the catheter;
forming an indicator notch on the catheter at a distance “h” from the distal end of the catheter, wherein the indicator notch indicates a proper insertion of the distal end of the catheter into a selected body cavity;
inserting the catheter into selected body cavity through an incision of the epidermis of a patient;
stopping the insertion of the catheter when the indicator notch is flush with the epidermis of the patient;
inflating the inflatable disk from the first configuration to the second configuration;
moving the securing ring towards the distal end of the catheter, wherein the securing ring engages the indicator notch; and
draining the body cavity of the patient.

18. A method as recited in claim 17 further comprising the steps of:
deflating the inflatable disk from the second configuration to the first configuration;
moving the securing ring in the direction of the proximal end of the catheter; and
removing the catheter from the body cavity through the incision on the epidermis.

19. A method as recited in claim 18 further comprising the steps of:
providing an inflation line, wherein the inflation line extends from an inflation pump through the lumen of the catheter to the inflatable disk;
providing a drainage line, wherein the drainage line extends from a fluid collection chamber to the distal end of the catheter to drain the body cavity; and
connecting a suctioning device to the drainage line, wherein the suctioning device is provided to suction fluid from the body cavity through the drainage line and into the fluid collection chamber.

20. A method as recited in claim 16 wherein the catheter is formed with an insert having a length “l,” wherein the insert extends from the distal end of the catheter towards the proximal end of the catheter, and wherein the insert promotes drainage of the body cavity.
